(19) United States
(12) Patent Application Publication
(10) Pub. No.: US 2024/0350385 A1
(43) Pub. Date: Oct. 24, 2024

(54) POLYMER FILM ANTIPERSPIRANT AND GRIP ENHANCER

(71) Applicant: Miller Biosciences LLC, St. Louis, MO (US)

(72) Inventors: Mark J. Miller, Olivette, MO (US); Seonyoung Kim, Olivette, MO (US)

(21) Appl. No.: 18/641,030

(22) Filed: Apr. 19, 2024

Related U.S. Application Data
(60) Provisional application No. 63/575,432, filed on Apr. 5, 2024.
(60) Provisional application No. 63/460,896, filed on Apr. 21, 2023.

(51) Int. Cl. A61K 8/40; A61Q 15/00

(52) U.S. Cl. CPC A61K 8/40; A61Q 15/00; A61K 2800/874; A61K 2800/95

(57) ABSTRACT

A cyanoacrylate polymer film-forming solution and treatment for inhibiting sweat and enhancing grip performance. The solution includes a polymer film-forming compound that, once formed into a film, dries the surface of the skin, blocks sweat pores to inhibit sweating without leaving a toxic byproduct or burdensome residue and enhances grip performance by reducing slippage and improving grip texture. The treatment includes a method of applying the cyanoacrylate polymer film-forming solution to the desired location, rubbing solution into a desired location, allowing the solution to dry, and repeating until desired effect is achieved.

### CROSS-REFERENCE TO RELATED APPLICATION

This application claims the benefit of U.S. Provisional Application No. 63/460,896, filed Apr. 21, 2023, and U.S. Provisional Application No. 63/575,432, filed Apr. 5, 2024. The entire disclosures of which are hereby incorporated by reference.

### BACKGROUND OF THE INVENTION

### Field of the Invention

This disclosure relates to the field of dermatological care. In particular, it relates to a treatment for hyperhidrosis, inhibiting perspiration, and enhancing grip security.

### Description of the Related Art

Every human being, from infants to the elderly, perspires or sweats. Perspiration or sweating is a normal physiological response to increased body temperature, environmental heat and humidity, emotions, nervousness, or physical exertion. Perspiration occurs when sweat is released from sweat glands, travels through sweat ducts and exits sweat pores to coat the skin's surface. The evaporation of sweat from the skin dissipates heat and is the primary thermoregulatory mechanism used by humans and primates.

The human body has three main types of sweat glands: eccrine, apocrine, and apoeccrine. Eccrine sweat glands are the most numerous of the three types and are responsible for the highest volume of sweat excretion. These sweat glands are coiled, tubular glands present on nearly the entire surface area of the body. The highest density areas of eccrine sweat glands on the human body are located on the palms of the hands and soles of the feet. In response to increased body temperature, the hypothalamus stimulates eccrine sweat glands via sympathetic innervation and the binding of acetylcholine muscarinic receptors to induce sweating.

In contrast to the widely distributed eccrine sweat glands, apocrine and apoeccrine sweat glands are limited to specific areas of the body and contribute much less to overall sweat production. Apocrine sweat glands are large, branched glands, located mainly in the axilla, breasts, face, scalp, and groin regions. Apocrine sweat glands are regulated by norepinephrine and are thus sensitive to strong emotions such as stress, fear, and sexual arousal. Apocrine sweat ducts do not directly penetrate the epidermis but connect to hair follicles, which releases sweat onto the skin's surface. The sweat produced from these sweat glands is more protein-rich than compared to watery eccrine sweat and produces more odor in the presence of bacteria.

The third category of sweat glands, apoeccrine sweat glands, share properties with both eccrine and apocrine sweat glands. These sweat glands are located in limited areas, like apocrine sweat glands, particularly only in the axillary region. Additionally, like eccrine sweat glands, apoeccrine sweat glands connect to and release sweat directly on the skin's surface.

While sweating undoubtedly evolved as an essential mechanism for regulating body temperature in hominids, sweating has unwanted consequences for modern humans, including making the hands and feet wet and slippery, stinging the eyes, increasing body odor, and soiling clothing. Additionally, conditions exist that cause an increase in sweating well beyond what is required for regulating body temperature.

Hyperhidrosis and bromhidrosis are among these conditions. Hyperhidrosis is excessive sweating beyond what is required for maintaining body temperature homeostasis. There are two types of hyperhidrosis: primary hyperhidrosis and secondary hyperhidrosis. Primary hyperhidrosis often begins in childhood and may result in episodes of excessive sweating at least once per week. This type of hyperhidrosis is idiopathic and affects the palmar, plantar, axillary, or craniofacial regions bilaterally. Primary hyperhidrosis etiology involves overactivity of otherwise normal sweat glands, is caused by faulty nerve signals triggering eccrine sweat glands to become overactive, and is typically associated with genetic predisposition.

Secondary hyperhidrosis is generalized or unilateral in distribution and typically begins in adulthood. This type of hyperhidrosis is less common compared to primary hyperhidrosis and is often a side effect of a physiological condition, medication, or an underlying pathology. Conditions that may cause secondary hyperhidrosis include: diabetes; menopause; thyroid disease; some types of cancer; nervous system disorders; and infections. Secondary hyperhidrosis may cause sweating all over the body and may even cause an increase in sweat production while sleeping.

Hyperhidrosis, either primary or secondary, is a common complaint among dermatology patients and may affect up to 5% of the US population and up to 18% of the global population. This condition can interfere with professional activities, such as using touch screens, shaking hands or putting on or changing gloves in a healthcare or restaurant setting. Additionally, there may be other negative impacts on daily activities, which may cause significant stress, limit social interactions, and reduce the quality of life for patients.

Similar to, and in some instances associated with, hyperhidrosis, is bromhidrosis. Bromhidrosis is a disorder that causes an excessive or abnormal odor to emanate from the skin and is caused by decomposition by bacteria and yeasts of sweat gland secretions and cellular debris. This condition could be the result of poor hygiene, infections, diet, medications, or may be associated with a genetic predisposition. Bromhidrosis affects all age groups, races, and sexes, but typically develops after puberty and generally has a male predominance. Bromhidrosis may involve either apocrine or eccrine sweat glands and may be associated with malodorous perspiration. Additionally, bromhidrosis may be chronic and can substantially impact a person's quality of life. Data indicates up to 3% of the US population is affected by bromhidrosis. Unfortunately, the incidence of bromhidrosis globally is unclear.

Because hyperhidrosis, both primary and secondary, and bromhidrosis are widely underreported and undertreated, the ability to know the full extent of the US and global population impacted by these conditions is severely limited. However, based on current data available and referenced in the preceding paragraphs, up to 8% of the US population and at least up to 18% of the global population is impacted by either hyperhidrosis or bromhidrosis.

Current treatment options for hyperhidrosis and bromhidrosis are limited and are often ineffective, expensive and can have significant side effects. The options include topical antiperspirant treatments, oral medicines, BOTOX® injections, iontophoresis and various surgical procedures designed to ablate the sweat glands. Additionally, palmar and plantar hyperhidrosis treatments are limited to only two FDA-approved options: BOTOX® injections and iontophoresis.

The most frequent and effective topical treatments for mild to moderate cases of these conditions are aluminum chloride formulations (e.g., aluminum chloride hexahydrate) due to their over-the-counter availability, low costs, and easy application. Aluminum chloride hexahydrate concentrations range from 6.25% to 12.5% in over-the-counter antiperspirants, while prescription formulations can be as high as 20%.

The antiperspirant mechanism is believed to be the result of aluminum salts interacting with sweat mucopolysaccharides and forming precipitates that block the eccrine sweat gland duct lumen. Topical aluminum chloride antiperspirants provide high satisfaction rates for decreasing axillary sweating, but show reduced efficacy for plantar and palmar hyperhidrosis.

Moreover, aluminum chloride antiperspirants work best when applied to clean skin at bedtime, remaining on the skin for 6 to 8 hours before being washed off with the process being repeated every 24 to 48 hours for 1-2 weeks to achieve clinical efficacy. However, skin irritation occurs in 21% of patients and is correlated with the higher aluminum chloride concentrations used in prescription-strength formulations.

Anticholinergic agents, such as glycopyrrolate and glycopyrronium, have also been widely used to treat hyperhidrosis. For instance, the use of topical glycopyrrolate has been effective in treating craniofacial hyperhidrosis and topical glycopyrronium wipes have been approved by the Food and Drug Administration (FDA) for axillary hyperhidrosis. These anticholinergic agents may be used in conjunction with antiperspirants or unilaterally.

Both glycopyrrolate and glycopyrronium work to reduce sweating by blocking the acetylcholine receptor on eccrine glands to inhibit sweating. One study found that at four weeks of treatment, 60% of patients reported reduced sweating compared to 20% in the sham group. Side effects reported include dry mouth, blurred vision, application-site pain, nasopharyngitis, and mydriasis, and of concern, 8% of patients discontinued use due to adverse events.

Injections of onabotulinum toxin A (BOTOX®) are FDA approved for treating severe axillary hyperhidrosis. BOTOX® treatment acts by blocking the nerve signals that trigger perspiration. BOTOX® injections can reduce sweat production by 50% for six months or longer. One study reported an approximately 90% decrease in sweat production at two weeks and 65% at twenty-four weeks, with 98% of research subjects recommending the therapy. Studies suggest common side effects include injection-site pain (sometimes severe) and itching, headache, muscle soreness, and increased compensatory facial sweating. Two pilot studies indicate that topical BOTOX® may also reduce sweating by 20-50% for up to six weeks. BOTOX® injections for palmar hyperhidrosis show modest efficacy with sweat reduction of 25-50% for three weeks to six months.

Aside from topical antiperspirants, BOTOX® injections and anticholinergic agents are the only drugs approved by the FDA for treating hyperhidrosis. Alternatively to drugs treating the condition, various procedures are FDA approved or are being developed for treating hyperhidrosis. However, each of these procedures comes with various disadvantages.

Iontophoresis is an FDA-approved procedure for treating palmar and plantar hyperhidrosis that exposes the skin to direct electrical current using a tap water bath device. The precise mechanism of action is unclear but various theories exist including mechanical obstruction, an electrochemical gradient blocking sweat secretion, inhibition of nerve transmission, and cytotoxic accumulation of hydrogen ions.

Iontophoresis requires 20-minute sessions repeated six to fifteen times over several weeks to be effective. After completing treatment, symptom improvement is seen in 80-85% of palmar/plantar hyperhidrosis patients. Reported side effects include skin dryness, irritation, erythema, and blistering. Despite the side effects, evidence from multiple trials suggests that tap water iontophoresis effectively treats palmar hyperhidrosis with acceptable patient satisfaction.

Other experimental hyperhidrosis treatment procedures are in the early experimental and developmental stages. Fractional microneedle radiofrequency (FMR) is a new procedure that delivers thermal energy to the skin using microneedles to ablate apocrine and eccrine sweat glands without destroying the epidermis. However, a blinded study could not conclusively demonstrate treatment efficacy or long-term safety of these experimental treatment procedures.

In a different cutting-edge procedure, 800 nm diode laser light was evaluated for treating axillary hyperhidrosis. However, the study did not show a significant decrease in sweating on laser-treated compared to untreated skin, and skin biopsies failed to show laser-induced skin changes.

Another procedure in development for hyperhidrosis treatment is micro-focused ultrasound. Micro-focused ultrasound produces thermal lesions or thermal coagulation points within the dermis, which is hypothesized to effectively damage the sweat glands while leaving the skin undamaged. This treatment method is also hypothesized to have long-term effects due to sweat glands having limited to no capacity for regeneration; however, since only two small studies have been reported, conclusions regarding efficacy or safety cannot be drawn.

A more invasive procedure, liposuction curettage, has been shown to reduce axillary hyperhidrosis sweat by 30-80% for six months or longer. However, side effects are numerous and long-lasting. Likely side effects include local pain and adverse events such as lasting neuropathic pain, hyperpigmentation, scar formation, local hair loss, seroma, infection, bleeding, hematoma, and skin necrosis.

Finally, MiraDry® is an innovative microwave therapy device that the FDA has cleared for treating primary axillary hyperhidrosis. This device focuses heat into the skin to ablate apocrine and eccrine sweat glands. However, only two case reports have been published, and there is concern that the procedure can cause ulnar and median nerve injury.

Although a variety of treatment options exist for hyperhidrosis, the available options typically require days to weeks to work, can negatively affect the surface texture of the skin, can be expensive and time-consuming, and have a variety of undesirable side effects and, in some cases, the risk of serious adverse events. Thus, a significant unmet need exists for new fast-acting, cheap, effective, and safe antiperspirants while enhancing the skin's texture, preventing blisters, and improving grip security.

One key issue with existing hyperhidrosis treatments is that they fail to address the need for improved skin surface texture and enhanced grip performance. In fact, antiperspirant treatments most often degrade the texture of the skin. For example, aluminum chloride makes the surface chalky, unsightly, and slippery, and iontophoresis can cause rough, irritated skin. Although these treatments may control sweating, their side effects significantly degrade palmar, plantar, and phalangeal grip function.

Unlike hyperhidrosis in axillary regions, which can be mitigated with absorbent pads and fabric choices, and hyperhidrosis in plantar regions, which can be mitigated with wicking socks or open sandal shoes, hyperhidrosis in palmar regions has no easy mitigation solution. Because there is no easy mitigation measure, the problem of excessive palmar sweating is especially difficult and burdensome for patients.

Sweaty palms interfere with various professional activities, including shaking hands, gripping writing utensils, using touch screens, gripping and working with tools, putting on or changing gloves in a healthcare or restaurant setting, or even typing. In addition to the professional environment, palmar hyperhidrosis can significantly affect recreational activities as well.

Sweaty hands make it difficult to securely grip a baseball, volleyball, football, or basketball. Additionally, sweaty palms in gymnastics can be seriously detrimental. Palmar hyperhidrosis can cause a gymnast's hands to slide during a floor tumbling routine, leading to potentially serious injuries, can make it difficult to grip the bars, rings, or beam during a bar or beam routine, and can lead to serious injuries in the event a gymnast's hands slip off the bars, rings, or beam in the middle of their routine.

In the case of racket sports, sweaty hands decrease grip security, leading to poor performance. Additionally, playing a racket sport with sweaty hands may cause players to compensate with excessive grip force, which can lead to blisters, wrist strain, arm strain, and other potential injuries.

Furthermore, because sweaty hands, feet, and phalanges are generally more prone to blister formation and torn skin, palmar and plantar hyperhidrosis can lead to pain and injury in an everyday context, or can lead to an impairment of athletic performance.

For example, someone who plays tennis regularly and consistently struggles with sweaty hands, may have difficulty gripping the racket securely, leading to overcompensating with excessive force and causing the development of recurring, painful blisters. Not only will the recurrent and painful blisters be likely, but the difficulty gripping the racket may also lead to the racket slipping out of the player's hands during play, especially during the tennis serve, which could damage the racket or, even worse, injure a bystander. Moreover, playing tennis with sweaty hands, leading to the overcompensation of gripping the racket too tightly, may also cause stress injuries to the player's elbow and wrist.

Additionally, talcum powder, silica-based and other specialized grip enhancers, and super-strength aluminum chloride antiperspirants could each be used to address sweaty hands. However, none of these options provide adequate sweat control while also ensuring a secure grip.

Alternatively, using a cyanoacrylate, like superglue or New-Skin® liquid bandages, on fingertips and hands has been known to toughen skin and repair cuts. However, these too come with a plethora of undesirable problems.

Notably, many cyanoacrylates commonly available, like superglue, release toxic byproducts, cure either too quickly or not quickly enough, have undesirable odors, can cause skin irritation, can result in fingers sticking together unintentionally, are difficult to remove, or have other undesirable side effects. Moreover, these commonly available products, like New-Skin®, typically are not water or sweat resistant, have a strong odor, require reapplication multiple times, and leave an aesthetically unpleasing peeling residue on the hands of the user.

While some available cyanoacrylates do not exhibit the above-mentioned problems, such as some cyanoacrylate tissue adhesives used in medical applications, these products are generally extensively used in the medical profession and not readily accessible to the general public. For instance, research immunologists performing animal surgeries in veterinary settings use Vetbond™ (3M), a tissue adhesive containing cyanoacrylate that is considered non-toxic when used on the skin of animals, during surgery.

Cyanoacrylate tissue adhesives have been used for decades to close wounds, stop bleeding, and prevent infection. Cyanoacrylates were one of the earliest compounds used for simple wound closure applications by soldiers in the 1950s. Cyanoacrylate tissue adhesives bond to the skin through Michael's addition reactions to tissue amines, forming durable but flexible films. Because of their reactivity to water, cyanoacrylates are stored in sealed bottles and sometimes contain hydroquinone stabilizers as used in Vetbond™. Many cyanoacrylate adhesives (such as n-Butyl-cyanoacrylate) can be applied directly to tissues without additional components, as the residual water and amines present on the tissue surface are sufficient to initiate polymerization, curing, and tissue bonding within seconds.

Due to their low toxicity and strong adhesion, n-Butyl and 2-Octyl cyanoacrylate are used in various settings, including thoracic, gastrointestinal, neurologic, cardiovascular, ophthalmologic, and vascular surgery. See Table 1, below. Cyanoacrylate tissue adhesives also provide an antimicrobial function and create a barrier that protects wounds from infection. Methyl and ethyl cyanoacrylates were the first formulations used in medicine, including abdominal and eye surgery. However, because methyl and ethyl cyanoacrylates were shown to release toxic degradation products into tissues, they are no longer used in the clinic.

TABLE 1
Approved Clinical Uses of Various Cyanoacrylate-Based Tissue Adhesives

| Trade Name | Manufacturer | Main Constituents | Approved Indication |
|---|---|---|---|
| Cyanoacrylates | Tissue Deal, | n-Butyl cyanoacrylate | Closure of topical skin incisions |
| Histoacryl, | LLC | | including laparoscopic incisions, and |
| Histoacryl Blue, | | | trauma-induced lacerations in areas of |
| and Histoacryl | | | low skin tension |
| Flexible | | | Microbial barrier |
| Dermabond | Ethicon | 2-Octyl cyanoacrylate | Topical application to hold closed |
| | | | easily approximated skin edges from |
| | | | surgical incisions, and trauma- |
| | | | induced lacerations |
| Indermil | Covidien LP | n-Butyl cyanoacrylate | Closure of topical skin incisions |
| | | | including laparoscopic incisions, and |
| | | | trauma-induced lacerations in areas of |
| | | | low skin tension |
| Trufill | Cordis | n-Butyl cyanoacrylate | Embolization of cerebral |
| | Neurovascular | and ethiodized oil | arteriovenous malformations when |
| | Inc. | | presurgical devascularization is |
| | | | desired |
| Omnex | Ethicon | 2-Octyl cyanoacrylate | For use in vascular reconstruction to |
| | | and butyl lactoyl | achieve adjunctive hemostasis by |
| | | cyanoacrylate | mechanically sealing areas of leakage |
| Glubran2 | GEM Italy | n-Butyl cyanoacrylate | For use in various surgeries, including |
| | | and | laparoscopic incisions and digestive |
| | | methacryloxysulpholane | tract endoscopy |
| | | | For use as an embolizing agent in |
| | | | radiology and vascular |
| | | | neuroradiology |
| Derma+Flex | Chemence | 60% of 2-Octyl | Topical application to hold closed |
| | Medical | cyanoacrylate and 40% | easily approximated skin edges from |
| | | of n-Butyl cyanoacrylate | surgical incisions, and trauma- |
| | | | induced lacerations |
| LiquiBand | LiquiBand | 2-Octyl cyanoacrylate | Closure of topical skin incisions |
| Exceed | Technology | | including laparoscopic incisions, and |
| | | | trauma-induced lacerations in areas of |
| | | | low skin tension |
| | | | Microbial barrier |
| SurgiSeal | Adhezion | 2-Octyl cyanoacrylate | Closure of topical skin incisions |
| | Biomedical, | | including laparoscopic incisions, and |
| | LLC | | trauma-induced lacerations in areas of |
| | | | low skin tensions |
| | | | Microbial barrier |

The two most common medical-grade formulations are n-Butyl and 2-Octyl cyanoacrylate, which have longer alkyl chains. These adhesives generate longer polymers that are more resistant to degradation and the release of toxic products such as formaldehyde and alkyl cyanoacetate. For example, Dermabond® is a topical tissue adhesive based on 2-Octyl cyanoacrylate that is stable enough to detach from the skin before degrading and releasing toxic degradation by-products. However, care must also be taken to prevent thermal damage and scarring of tissues since the cyanoacrylate polymerization reaction is exothermic.

Furthermore, although n-Butyl and 2-Octyl cyanoacrylate are considered harmless to most patients, they can cause allergic contact dermatitis in some cases when used to close surgical wounds, where a study reported 2.7% incidence for 2-Octyl cyanoacrylate and 2.2% for n-Butyl cyanoacrylate. While the topical application of cyanoacrylates to unbroken skin will likely reduce the risk of allergic contact dermatitis, this needs to be tested in clinical safety trials.

However, even these cyanoacrylates are imperfect for grip enhancement and inhibiting perspiration. As discussed above, the polymerization reaction of these cyanoacrylates is exothermic, which could cause injury to the user. Additionally, there is a risk of unintentional skin bonding and allergic contact dermatitis that may occur with the use of these cyanoacrylates. Further, while some cyanoacrylates are used for various medical applications, as shown in Table 1, there currently are no cyanoacrylates formulated for use as antiperspirants or grip enhancers.

Thus, an improvement in cyanoacrylates and similar polymer film-forming agents which allow them to be available to the general public and used commercially for sweat control and grip enhancement, while eliminating or greatly reducing the risk of injury and potential for allergic contact dermatitis, would be beneficial for the public who suffer from hyperhidrosis or other conditions that cause increased perspiration.

### SUMMARY OF THE INVENTION

Because of this and other problems in the art, described herein, is a topical film-forming agent, particularly a polymer film-forming agent, and associated method of using such that, when the film-forming agent is spread over the surface of the skin, it blocks sweat pores, prevents sweating, and enhances gripping ability while preventing blisters, reducing or eliminating the risks of releasing toxic byproducts, causing skin irritation, or unintentional skin bonding.

Described herein, among other things, is a topical polymer film-forming solution and treatment method that inhibits sweat and enhances grip performance without causing irritation, releasing toxic by-products, or leaving burdensome residue that may interfere with grip performance or be aesthetically unpleasing to look at. The solution includes a polymer film-forming compound that, once formed into a film, blocks a large number of sweat pores to inhibit sweating without leaving a toxic byproduct or burdensome residue and enhances grip performance by reducing slippage and improving grip strength. The treatment includes a method of applying the cyanoacrylate polymer film-forming solution to the desired location, rubbing solution into a desired location, allowing the solution to dry, and repeating until desired effect is achieved.

The polymer film-forming solution of the present disclosure is non-toxic and spreadable on skin surfaces and non-skin surfaces. The polymer film-forming solution comprises: a polymer film-forming agent; and a stabilizer, wherein the stabilizer inhibits spontaneous polymerization of the polymer film-forming agent; wherein the polymer film-forming agent and the stabilizer are combined into a polymer film-forming compound; wherein the compound cures into a film less than 20 μm thick in about 10-15 seconds with manual friction applied or in about 20-30 seconds by air drying; wherein after the compound cures into the film, the compound broadly blocks sweat pores and increases skin surface friction.

In embodiments, the polymer film-forming agent is a cyanoacrylate.

In some embodiments, the cyanoacrylate is selected from the group consisting of: n-Butyl-cyanoacrylate, 2-Octyl cyanoacrylate, Octyl cyanoacrylate, Isobutyl cyanoacrylate, Methyl cyanoacrylate, Ethyl cyanoacrylate, and combinations thereof.

In some embodiments, the stabilizer is selected from the group consisting of: <1% hydroquinone, Mequinol, S02, Butylated hydroxyanisole, and combinations thereof.

In other embodiments, the polymer film-forming solution further comprises a non-toxic dye additive.

In other embodiments, the polymer film-forming solution further comprises a perfume additive.

In other embodiments, the polymer film-forming solution further comprises a medicament additive.

In other embodiments, the polymer film-forming solution further comprises a viscosity modifier additive.

Also discussed herein is a method of using a topical polymer film-forming solution, the method comprising: providing the polymer film-forming solution of this disclosure; applying about 100-200 μl of the solution on a target area of human skin; spreading the solution to form a thin film over the target area; and curing the thin film, wherein the cured film allows an unimpeded sense of touch, feel, and dexterity, inhibits perspiration, prevents blisters, increases skin surface friction, and improves grip security in said target area.

In embodiments, the target area comprises a user's hands, feet, phalanges, axillary, pubic, or craniofacial area.

In further embodiments, the target area is a non-skin surface.

In other embodiments, the cured film reduces the occurrence of sweat-associated blistering, tearing, and epidermal detachment in the target area.

In some embodiments, perspiration is inhibited for at least one hour and up to twenty-four hours.

In some embodiments, the film is cured by applying manual friction to the target area for about 10-15 seconds.

In other embodiments, the film is cured by air drying the target area for about 20-30 seconds.

In further embodiments, the 100-200 μl of the solution is applied by applying multiple drops, about 20 μl each, on the target area and then spreading the solution over said target area.

In yet other embodiments, the solution is applied to the target area via a spray bottle.

In yet other embodiments, the solution is applied to the target area via a roll-on applicator.

In yet other embodiments, the solution is applied to the target area via crushable capsules containing the solution.

In still further embodiments, the solution is applied to target area via a bag having the solution inside; wherein the user places at least one finger on the outside of the bag and inverts the bag over said at least one finger, such that the solution is on the outer surface of said bag and exposed to air, then said user spreads the solution by rubbing the outer surface of the bag over the target area.

### BRIEF DESCRIPTION OF THE DRAWINGS

FIG. 1A shows human skin, eccrine sweat glands, and apocrine sweat glands, untreated by the disclosed cyanoacrylate polymer film-forming solution.

FIG. 1B shows the human skin of FIG. 1A a polymer layer formed on the surface of the skin following topical application of the disclosed cyanoacrylate polymer film-forming solution.

FIG. 2 depicts embodiments of application methods of the cyanoacrylate polymer film-forming solution of the present disclosure.

FIG. 3 depicts alternative embodiments of application methods of the cyanoacrylate polymer film-forming solution of the present disclosure.

FIG. 4 depicts an additional alternative embodiment of an application method of the cyanoacrylate polymer film-forming solution using a plastic bag finger cot applicator.

FIG. 5 depicts artificial sweat present on the surface of nitrile gloves after spreading the cyanoacrylate polymer film-forming solution on the nitrile gloves surface and the results of a grip efficiency test after the cyanoacrylate polymer film-forming solution of the present disclosure was applied compared to results without the presence of the cyanoacrylate polymer film-forming solution of the present disclosure.

FIG. 6 depicts the results of a grip efficiency test after the application of the disclosed cyanoacrylate polymer film-forming solution, which demonstrates application of the disclosed cyanoacrylate polymer film-forming solution results in enhanced gripping performance.

### DETAILED DESCRIPTION OF THE PREFERRED EMBODIMENT(S)

The following detailed description and disclosure illustrates by way of example and not by way of limitation. This description will clearly enable one skilled in the art to make and use the disclosed systems and methods, describes several embodiments, adaptions, variations, alternatives and uses of the disclosed systems and methods. As various changes could be made in the systems and methods described in this disclosure without departing from the scope of the disclosure, it is intended that all matter contained in the description or shown in the accompanying drawings shall be interpreted as illustrative and not in a limiting sense.

The film-forming agent of the present disclosure may be a cyanoacrylate, however, other polymer film-forming agents are possible. Particularly, other polymer film-forming agents that are spreadable and non-toxic (i.e., not poisonous or not containing poisonous substances, as defined by Cambridge Dictionary, https://dictionary.cambridge.org/us/dictionary/english/non-toxic, which is herein incorporated by reference), which are thin enough to not preclude a user's sense of touch or feel, have bonding or curing times as discussed in the present disclosure, and cover the sweat pores, as discussed below, could be used in place of a cyanoacrylate in a non-toxic, polymer film-forming agent as discussed herein. Hereinafter, where the present disclosure refers to the polymer film-forming agent as a cyanoacrylate, it should be understood that the term cyanoacrylate is being used generally. It should be further understood that the term “cyanoacrylate,” as used in this disclosure, means any polymer film-forming agent that exhibits the properties and characteristics mentioned in this paragraph and described in detail below.

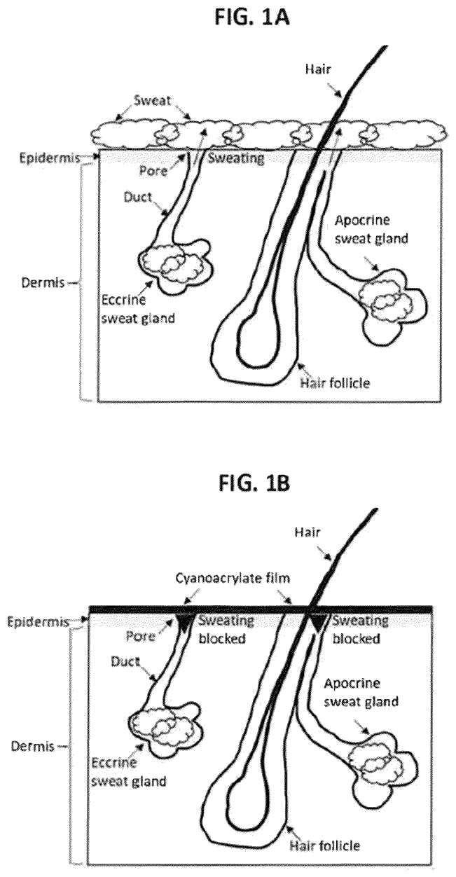

FIGS. 1A and 1B show the skin surface before and after treatment with a topical polymer film-forming solution, particularly, a cyanoacrylate solution. As depicted in FIG. 1A, before cyanoacrylate solution application, eccrine sweat glands secrete sweat onto the epidermal surface of the skin. This is because sweat produced in the sweat glands travels up the sweat ducts and is released through pores onto the surface of the skin, as seen in FIG. 1A. The sweat on the surface of the skin results in the skin becoming slippery and wet.

In contrast, as depicted in FIG. 1B, after the topical cyanoacrylate solution is applied and cured, the surface of the skin is dry. This is because the cured cyanoacrylate solution creates a thin, transparent film on the epidermis, which blocks sweat pores and effectively inhibits perspiration. The topical cyanoacrylate solution of this disclosure serves as an antiperspirant, as described in detail further herein, and also improves skin surface texture, enhances grip performance of the areas applied thereto by increasing skin surface friction, prevents sweat-associated skin blistering and tearing.

The topical cyanoacrylate treatments of this disclosure are fast-acting, effective, affordable, accessible, and safe. Further, the cyanoacrylate solution of this disclosure may significantly improve the quality of life for hyperhidrosis and bromhidrosis patients, improve the workflow and reduce the stress of healthcare professionals, restaurant workers, and others who are required to wear gloves, and may also benefit laborers and sports enthusiasts by increasing grip security while reducing the risk of blisters, tears, and grip associated stress injuries.

Certain embodiments of the present disclosure include the application of a thin layer of a polymer film-forming solution containing a polymer film-forming compound, which will typically comprise, consist of, or consist essentially of a cyanoacrylate (typically in amounts >90%) and a stabilizer (such as, for example, <1% hydroquinone, Mequinol, S02, Butylated hydroxyanisole, or other ingredients, such as described in U.S. Pat. No. 6,512,023 and discussed in Polymeric Tissue Adhesives, Sungmin Nam and David Mooney, Chem. Rev. 2021, 121 (18), 11336-11384, the entire disclosures of both are herein incorporated by reference) to inhibit spontaneous polymerization of the cyanoacrylate and improve shelf life. In a preferred embodiment, a non-toxic dye may be added to the solution to facilitate visualization and promote even application on the skin.

In the present disclosure, cyanoacrylate tissue adhesives such as n-Butyl-cyanoacrylate and 2-Octyl cyanoacrylate are preferred as the cyanoacrylate in the polymer film-forming compound due to their low toxicity and slower polymerization rates. However, these cyanoacrylates are not required and other cyanoacrylates, as discussed herein, may be used, either singularly or in combination.

For instance, Octyl cyanoacrylate, Isobutyl cyanoacrylate, Methyl cyanoacrylate, Ethyl cyanoacrylate, and other related molecules could be used if polymerization rate and toxicity issues were addressed through chemical modifications to the structures or by adding stabilizers and/or polymerization modifiers to the solution. Additionally, other polymer film-forming agents, which share the same traits or exhibit similar traits to the aforementioned cyanoacrylates, could also be used, such as the various chemistries as summarized in Polymeric Tissue Adhesives, Nam et al, which has been incorporated by reference.

Specifically, as summarized in Nam, the following alternative functional groups and chemistries could be used to produce topical polymer film-forming antiperspirants: NHS esters, Aminolysis and thiolysis; Aldehydes, Schiff-base reaction, Hemithioacetal formation 1,2-aminothiol, Thiazolidine formation; Isocyanates, Michael-type addition (Urea linkage); Catechol, Quinone formation by oxidation, and subsequent Michael addition or Schiff-base reaction; and Transglutaminases, Amide bond formation. However, this list is illustrative only and other functional groups and chemistries may also be suitable embodiments. For example, other polymer film-forming agents that may be used in accordance with the present disclosure are those that can be formulated to be easily spread over the skin, seal sweat pores before curing into a film, do not restrict the sense of touch or feel of the user, do not inhibit dexterity of the user, and increase the grip function of the target area, such as the hands, feet, or phalanges of the user.

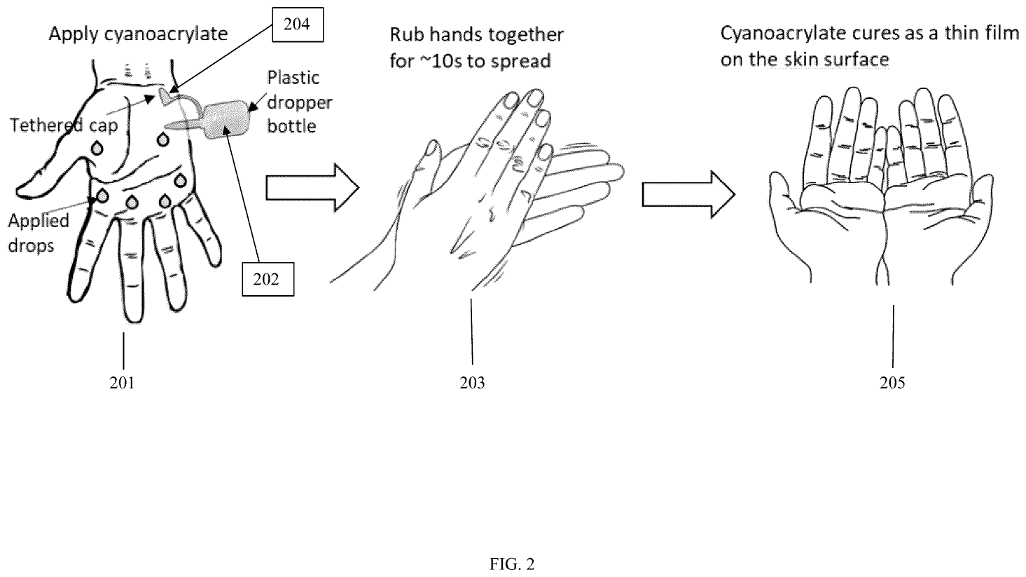

As depicted in FIG. 2, users apply approximately 100-200 μl of the disclosed cyanoacrylate solution in multiple drops (approximately 20 μl each) on the palm of the hands (201), or other target area of treatment. However, these volumes are merely illustrative and other volumes of the disclosed cyanoacrylate solution can be used. After applying the drops over the target area, the user spreads the solution over the target area and applies manual friction to the target area, e.g., rubs the hands and fingers together (203), for approximately ten to fifteen seconds to create a thin transparent film of cured cyanoacrylate adhesive (205) over the target area. The film of cyanoacrylate then blocks sweat pores and inhibits perspiration as seen in FIG. 1B. This application of the cyanoacrylate solution leaves the palm and phalangeal surface of the skin with a satin-like texture that enhances grip function.

In an embodiment, the cyanoacrylate is in a small plastic or glass bottle with approximately 3-5 ml of solution capacity (202), however, this volume is illustrative and plastic or glass bottles with other volumes are possible. The plastic or glass bottle (202) is also equipped with an integrated dropper mechanism, which allows for the controlled delivery of multiple drops to the skin. Dispersing the drops over the target area makes it easier to quickly spread the cyanoacrylate solution over the skin surface by hand before it cures. This method of application of the cyanoacrylate is simple and straightforward and could be used to create thin cyanoacrylate films on various surfaces, including palmar skin and phalangeal creases (201), the entire foot (301), (303), or (305), or other skin surfaces such as the armpits (307), (309), or (311) or forehead, for example. Gloves may be worn while hand-spreading the solution to prevent contact with the hands if desired.

The container of cyanoacrylate (202) may also contain a tethered cap or finger ring (204), which may allow the container (202) to be secured on one finger while the adhesive is spread over the skin. This may facilitate application of the cyanoacrylate by permitting the user to spread the solution without having to set the bottle down, which could delay spreading the solution by hand. However, this application method is by no means required and in other embodiments, the application methods include spray bottles (305) and (307), roll-on applicators (309), pieces of material containing the solution (311), or crushable capsules containing the solution.

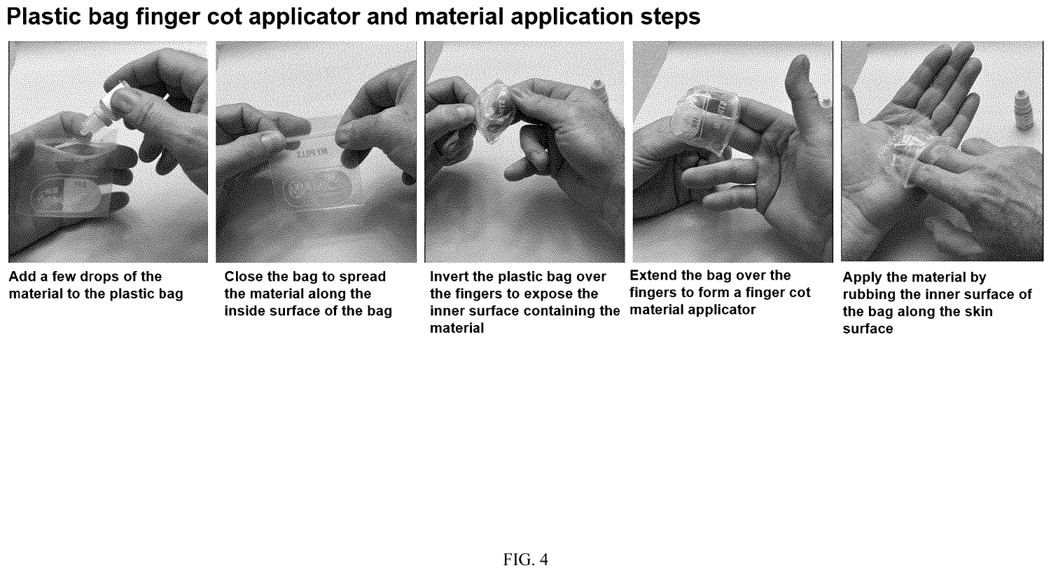

Additionally, as depicted in FIG. 4, plastic or foil bags containing the cyanoacrylate solution could be used for the application of the disclosed cyanoacrylate compound. The application method of the plastic or foil bags includes the user placing at least one finger on the outside of the bag containing the solution, then inverting the bag over the at least one finger. When the bag is inverted over the at least one finger, the cyanoacrylate solution that was on the inside of the bag becomes on outer surface of the bag, such that the cyanoacrylate solution is exposed to the air and can be applied. Because the disclosed cyanoacrylate solution preferably takes about 20-30 seconds to cure when exposed to air (i.e., air drying), which is longer than the cyanoacrylate solution would preferably take to cure when manual friction is applied, this method of application permits the user to spread the solution over the target area without risk of the solution curing on the surface of the bag prior to application. To apply the solution to the target area, the user uses the bag as a finger cot to rub the outer surface of the bag on the target area, which spreads the solution over the target area. See FIG. 4.

Regardless of the application method, when the disclosed cyanoacrylate solution is applied to the target area, the cyanoacrylate film may improve the texture of the skin in the target area of which the cyanoacrylate is applied, e.g., the palmar, plantar, and phalangeal skin, compared to aluminum chloride-based antiperspirants, which leave a slick, greasy residue that degrades grip function. Additionally, the cyanoacrylate solution of the present disclosure may be applied to non-skin surfaces to improve grip performance of the non-skin surface, such as, for example, applying the cyanoacrylate solution to a glove to improve the grip performance of the glove. Furthermore, the skin texture and grip-enhancing effects of cyanoacrylate films on the target area may last for up to twenty-four hours or more, depending on the extent of perspiration, water exposure, and frequency of washing.

The cyanoacrylate of this disclosure may be applied multiple times, if necessary, or may be removed by the user when desired. If the skin begins to sweat after the initial treatment, the skin may be merely wiped off and cyanoacrylate film reapplied to prevent further sweating. However, the skin is not required to be wiped off and in some embodiments, applying the disclosed cyanoacrylate solution to damp skin may enhance its performance by quickly drying the skin surface and preventing further perspiration to result in the disclosed cyanoacrylate providing a stronger grip for the user. Conversely, the film can be removed by washing with an exfoliating brush and soapy water or using nail polish remover, makeup remover solutions/wipes or sanitizing cleaning wipes.

The application of cyanoacrylate solutions could also be used to inhibit perspiration on other skin surfaces, including but not limited to axillary, pubic, and craniofacial areas. The efficacy and durability of the antiperspirant effect at these different sites may depend on variables, such as whether sweat is being produced by eccrine or apocrine glands, the level of sweat production, and how often the site is washed.

In the case of bromhidrosis, the anti-microbial activity of cyanoacrylate may also help reduce the unpleasant odor that results from bacterial interactions with apocrine sweat. Cyanoacrylate compounds used as antiperspirants may also be advantageous because cured cyanoacrylate films are transparent and will not rub off on clothing, unlike aluminum chloride antiperspirants that leave a residue on the axillary skin that is both unsightly and can stain or discolor clothing.

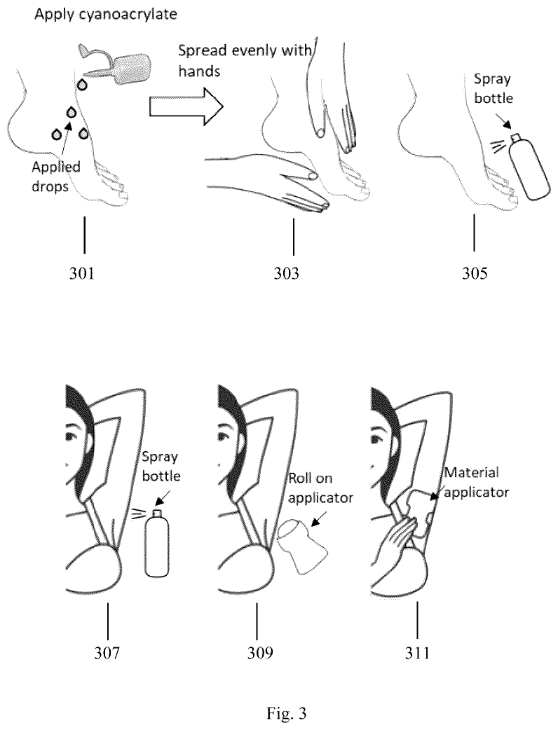

The cyanoacrylate compound typically cures over about 10-15 seconds with manual friction applied, such as hand rubbing (201), (203), and (205), as depicted in FIG. 2, or massaging the solution onto the skin. However, if hand application is not desirable, the cyanoacrylate solution may be applied using other methods and devices, as seen in FIG. 3, including a spray bottle (305) and (307), a roll-on applicator (309), or a piece of material containing the solution such as wipes (311).

The application of the cyanoacrylate solution using these alternative application methods permits the solution to be applied to larger surfaces, which may be desirable. These application methods permit the cyanoacrylate solution to be applied evenly to the skin as a thin film, which then cures. This thin, evenly spread, cured film is key to its mechanism of action as an antiperspirant.

These alternative application methods may also be combined with spreading by hand, or gloves could be worn to prevent the cyanoacrylate solution from coming into contact with the skin of the hands, such that manual friction (i.e., rubbing the solution in by hand, using an applicator device to rub or massage the solution in) is applied and the solution cures in about 10-15 seconds. Alternatively, the cyanoacrylate solution will cure after about 20-30 seconds of exposure to air if no manual friction is applied. Once the cyanoacrylate compound cures as a thin film and is bound to the epidermis, it may effectively block sweat pores and prevent sweating, despite the many variables associated with different skin regions of the body, e.g., axillary vs. plantar skin.

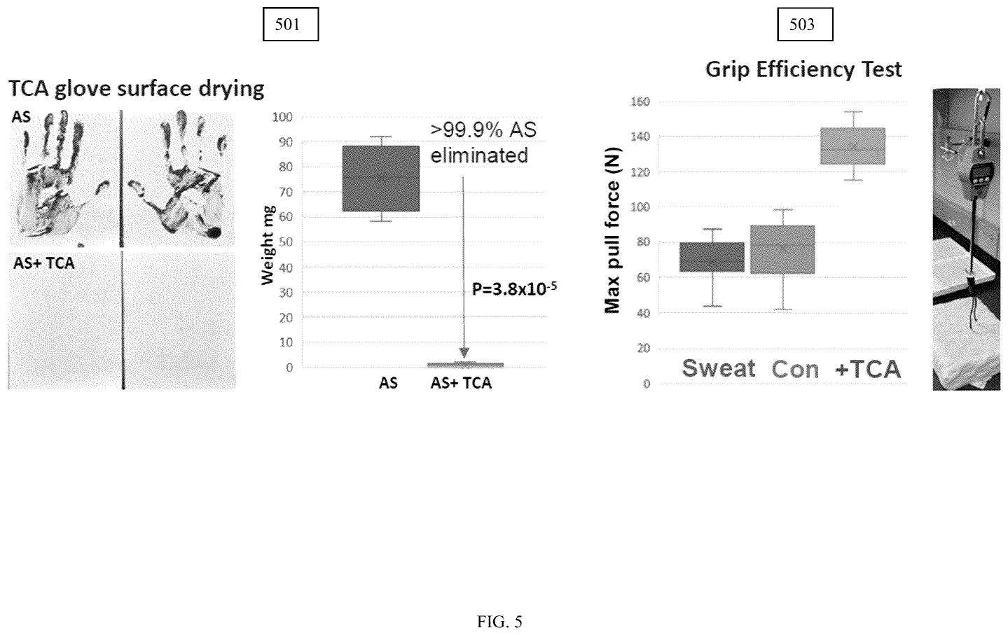

When the cyanoacrylate compound is applied and cured into a film, the surface where the cyanoacrylate film was applied is nearly completely eliminated of surface moisture. This is depicted in FIG. 5, where, after a nitrile glove was treated with cyanoacrylate for 15 seconds, the nitrile glove had 99.9% of artificial sweat (or moisture) eliminated (501). As further depicted in FIG. 5, when the cyanoacrylate is applied and cured, the resulting grip efficiency of the user is improved. Specifically, as seen in the Grip Efficiency Test (503) of FIG. 5, the max pull force with the cyanoacrylate applied is at least double the max pull force without the cyanoacrylate.

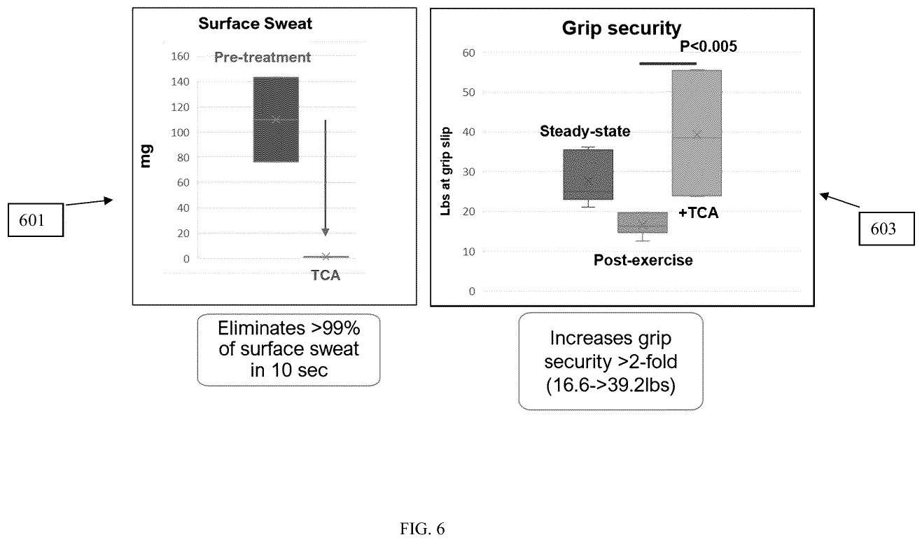

This enhanced grip strength is further illustrated in FIG. 6, which depicts the results of an experiment testing grip efficiency. This grip efficiency test demonstrates the grip security, which is shown in (603), increases more than two-fold with the cyanoacrylate solution applied. See FIG. 6. The grip efficiency experiment was performed by researchers using a small handheld crane scale with two handles, the two handles of the small handheld crane scale were then pulled in different directions, and when the grip began to slip, the grip force was recorded by the researcher. The results of the test were recorded and, as shown in the Grip Security (603) graph, demonstrate the cyanoacrylate solution results in enhanced grip performance.

Additionally, it is known that application of the cyanoacrylate solution inhibits sweating and blocks sweat pores (see Provisional App. No. 63/575,432, FIG. 8). Without being bound by any known process, it is believed that the cyanoacrylate solution works to inhibit sweating by blocking sweat pores and because the sweat in the dermis has sodium and other elements removed, leaving only water remaining, which the body does not recognize as needing to expel, there is no build up of pressure against the cyanoacrylate film. Based on this understanding, one possible way perspiration is inhibited is by the cyanoacrylate solution blocking the sweat pores, and because only water remains in the dermis and the body is not trying to expel the water, perspiration may be inhibited. See FIG. 1B. While the cyanoacrylate solution inhibiting perspiration may be the result of a process different than believed, the cyanoacrylate solution nevertheless inhibits perspiration.

Furthermore, following treatment with the cyanoacrylate solution of the present disclosure, no signs of sweat building up below the cyanoacrylate layer or visible irritation on the palmar skin were present even after vigorous exercise. This indicates that the disclosed cyanoacrylate does not cause skin irritation or other undesired effects. This may be due, in part, to the residual film left on the skin being so thin and non-intrusive. Based on testing, as discussed in Provisional Application No. 63/575,432, FIG. 7, the thickness of the film following application of the cyanoacrylate is about 10 μm. As such, in the present disclosure a film thickness of less than 10 μm is preferred, however, a film thickness of less than 15 μm, 20 μm, or 30 μm may also suffice for the present disclosure.

The current disclosure includes a method of inhibiting perspiration comprising, consisting of, or consisting essentially of a solution of cyanoacrylate, a polymerization stabilizer, and a method of topical application to create a film on the skin to block sweat pores and inhibit sweating. Other embodiments may include the addition of a non-toxic dye to visualize the solution and facilitate even application to the skin, but this is not essential to the mechanism of action and may be omitted for large-area application methods. In yet other embodiments, perfumes may be added to the cyanoacrylate solution if desired. In still further embodiments, other additives, such as plasticizers, viscosity modifiers, and medicaments (e.g., bug repellent, sunscreens, etc.) may be added to the cyanoacrylate solution. Also, various chemical stabilizers and polymerization modifiers may be added to the cyanoacrylate solution to improve its shelf life and/or to alter the polymerization kinetics to facilitate even spreading on the skin.

In addition to treating hyperhidrosis and bromhidrosis, there are additional applications that are logical extensions of the present disclosure based on the unique attributes of cyanoacrylate films, which set them apart from existing topical antiperspirants. For example, wearing disposable gloves is required for healthcare professionals and restaurant workers, but after the hands become sweaty it is difficult to change gloves. In such a case, topical cyanoacrylate antiperspirants could improve the workflow of millions of healthcare and restaurant workers by making it easier to put on and change gloves during their duties.

Furthermore, topical cyanoacrylate solution can be used to reduce the occurrence of sweat-associated skin blistering and tearing. This is a common problem with laborers, athletes, hikers, and soldiers, and the cyanoacrylate solution may be used to treat feet and hands to prevent blisters during work, hiking, exercise, or sports. When skin becomes moist and waterlogged due to sweating, it becomes more fragile and prone to tears, vesiculation (i.e., blistering), and epidermal detachment. Blistering can cause pain, emotional stress, and decrease physical performance. Because cyanoacrylate films both increase the structural integrity of the epidermis to abrasion and keep the skin dry, they may be effective at reducing sweat-associated skin blisters, tears on the hands, fingers, and feet, and abrasion-associated skin damage.

Additionally, topical cyanoacrylate films enhance skin texture and increase skin surface friction, thus improving grip function and strength of the target area to which the cyanoacrylate solution was applied. Topical cyanoacrylate may significantly enhance sports performance when playing racket sports and other sports such as basketball, volleyball, football, and baseball that depend on securely gripping a ball. This increase in grip strength is depicted in FIG. 5, where the results of a grip efficiency test are shown (503). These test results show the max pull force of a testing apparatus without the cyanoacrylate treatment compared to with the cyanoacrylate treatment, and indicate the pull force with the cyanoacrylate treatment is significantly higher than without (503).

In addition to improving overall performance, the cyanoacrylate compound will reduce the risk of grip-related injuries due to grip slipping and/or excessive grip force. Furthermore, cyanoacrylate antiperspirant and grip enhancement characteristics may be beneficial to gymnasts, acrobats, and rock climbers to increase performance and safety. Finally, the texture and grip enhancement attributes of the invention may further benefit workers who use various tools daily such as hammers, saws/knives/shears, and wrenches, as well improve the gripping of weapons such as swords, staffs, spears, nun chucks, handguns, and rifles.

The qualifier “generally,” and similar qualifiers as used in the present case, would be understood by one of ordinary skill in the art to accommodate recognizable attempts to conform a device to the qualified term, which may nevertheless fall short of doing so. This is because terms such as “perpendicular” are purely geometric constructs and no real-world component or relationship is truly “perpendicular” in the geometric sense. In the same way, as this disclosure discusses various volumes and other mathematical descriptors or terms of measurement, when the qualifier, such as, for example, “approximately,” is preceding the mathematical descriptor or term, it is not intended for that term to be precisely the number indicated. This is because variations from geometric and mathematical descriptions are unavoidable due to, among other things, manufacturing tolerances resulting in shape variations, defects and imperfections, non-uniform thermal expansion, natural wear, and calibration tolerances. Moreover, there exists for every object a level of magnification at which geometric and mathematical descriptors fail due to the nature of matter. One of ordinary skill would thus understand the term “generally” and relationships contemplated herein regardless of the inclusion of such qualifiers to include a range of variations from the literal mathematical meaning of the term in view of these and other considerations.

While the invention has been disclosed in conjunction with a description of certain embodiments, including those that are currently believed to be the preferred embodiments, the detailed description is intended to be illustrative and should not be understood to limit the scope of the present disclosure. As would be understood by one of ordinary skill in the art, embodiments other than those described in detail herein are encompassed by the present invention. Modifications and variations of the described embodiments may be made without departing from the spirit and scope of the invention.

It will further be understood that any of the ranges, values, properties, or characteristics given for any single component of the present disclosure can be used interchangeably with any ranges, values, properties, or characteristics given for any of the other components of the disclosure, where compatible, to form an embodiment having defined values for each of the components, as given herein throughout. Further, ranges provided for a genus or a category can also be applied to species within the genus or members of the category unless otherwise noted.

1. A non-toxic polymer film-forming solution for spreading on skin, the solution comprising:
a polymer film-forming agent; and
a stabilizer, wherein the stabilizer inhibits spontaneous polymerization of the polymer film-forming agent;
wherein the polymer film-forming agent and the stabilizer are combined into a polymer film-forming compound;
wherein the compound cures into a film less than 20 μm thick in about 10-15 seconds with manual friction applied or in about 20-30 seconds by air drying;
wherein after the compound cures into the film, the compound blocks sweat pores and increases skin surface friction.

2. The solution of claim 1, wherein the polymer film-forming agent is a cyanoacrylate.

3. The solution of claim 2, wherein the cyanoacrylate is selected from the group consisting of: n-Butyl-cyanoacrylate, 2-Octyl cyanoacrylate, Octyl cyanoacrylate, Isobutyl cyanoacrylate, Methyl cyanoacrylate, Ethyl cyanoacrylate, and combinations thereof.

4. The solution of claim 1, wherein the stabilizer is selected from the group consisting of: <1% hydroquinone, Mequinol, S02, Butylated hydroxyanisole, and combinations thereof.

5. The solution of claim 1, further comprising a non-toxic dye additive.

6. The solution of claim 1, further comprising a perfume additive.

7. The solution of claim 1, further comprising a medicament additive.

8. The solution of claim 1, further comprising a viscosity modifier additive.

9. A method of using a topical polymer film-forming solution, the method comprising:
Providing the polymer film-forming solution of claim 1;
applying about 100-200 μl of the solution on a target area of human skin;
spreading the solution to form a thin film over the target area;
curing the thin film, wherein the cured film allows an unimpeded sense of touch, feel, and dexterity, inhibits perspiration, increases skin surface friction, and improves grip security in said target area.

10. The method of claim 9, wherein the target area comprises a user's hands, feet, phalanges, axillary, pubic, or craniofacial area.

11. The method of claim 9, wherein the cured film reduces the occurrence of sweat-associated blistering, tearing, and epidermal detachment in the target area.

12. The method of claim 9, wherein perspiration is inhibited for at least one hour and up to twenty-four hours.

13. The method of claim 9, wherein the film is cured by applying manual friction to the target area for about 10-15 seconds.

14. The method of claim 9, wherein the film is cured by air drying the target area for about 20-30 seconds.

15. The method of claim 9, wherein the 100-200 μl of the solution is applied by applying multiple drops, about 20 μl each, on the target area and then spreading the solution over said target area.

16. The method of claim 9, further comprising applying the solution to the target area via a spray bottle.

17. The method of claim 9, further comprising applying the solution to the target area via a roll-on applicator.

18. The method of claim 9, further comprising applying the solution to the target area via crushable capsules containing the solution.

19. The method of claim 9, further comprising applying the solution via a bag having the solution inside;
wherein the user places at least one finger on the outside of the bag and inverts the bag over said at least one finger, such that the solution is on the outer surface of said bag and exposed to air, then said user spreads the solution by rubbing the outer surface of the bag over the target area.
